(19) United States
(12) Patent Application Publication
(10) Pub. No.: US 2026/0207183 A1
(43) Pub. Date: Jul. 23, 2026

(54) RADIAL COMPRESSION JOINT FOR BIOPSY NEEDLE OR GUIDEWIRE ASSEMBLY

(71) Applicant: BOSTON SCIENTIFIC SCIMED, INC., Maple Grove, MN (US)

(72) Inventors: Austin Grant JOHNSON, Worcester, MA (US); Barry WEITZNER, Great Barrington, MA (US)

(21) Appl. No.: 19/436,654

(22) Filed: Dec. 30, 2025

Related U.S. Application Data
(60) Provisional application No. 63/748,913, filed on Jan. 23, 2025.

(51) Int. Cl. A61B 10/02; A61L 29/02

(52) U.S. Cl. CPC A61B 10/0233; A61L 29/02

(57) ABSTRACT

A medical component includes first and second members and a tube formed of a material comprising shape memory properties. The tube has a memorized shape comprising an inner diameter less than an outer diameter of the first and second members, the tube positioned so that an inner surface of the tube is in abutting contact with an outer surface of a first portion of the first member and an outer surface of a first portion of the second member. The tube provides a radially compressive force to the outer surfaces of the first and second members to maintain the first member in position relative to the second member at least by a static friction.

### PRIORITY CLAIM

The present disclosure claims priority to U.S. Provisional Patent Application Ser. No. 63/748,913 filed Jan. 23, 2025; the disclosure of which is incorporated herewith by reference.

### BACKGROUND

Nickel-titanium alloys, commonly referred to as Nitinol, possess properties including a shape memory effect, superelasticity (or pseudoelasticity) and flexibility that make these alloys suitable for a variety of medical purposes. In one example, Nitinol components such as a needle and/or a catheter may be used for fine needle biopsy (FNB) to permit these components to navigate a tortuous path through body lumens to access target tissue through tight turning radii without plastic deformation. When conducting endoscopic ultrasound guided (EUS) or endobronchial ultrasound guided (EBUS) biopsy procedures, physicians find the target biopsy anatomy under endoscopic ultrasound and then pass the biopsy needle down the working channel of the scope. If the biopsy needle is not sufficiently flexible, the scope will deflect as the needle is passed therethrough, shifting the physician's view away from the biopsy target. Although a needle or catheter formed entirely of Nitinol may be sufficiently flexible to avoid this issue, the high cost of Nitinol is prohibitive for most such products.

### SUMMARY

The present disclosure relates to a medical component which includes a first member; a second member; and a tube formed of a material comprising shape memory properties. The tube has a memorized shape comprising an inner diameter less than an outer diameter of the first and second members, the tube positioned so that an inner surface of the tube is in abutting contact with an outer surface of a first portion of the first member and an outer surface of a first portion of the second member, the tube providing a radially compressive force to the outer surfaces of the first and second members to maintain the first member in position relative to the second member at least by a static friction.

In an embodiment, the tube is formed of Nitinol.

In an embodiment, the first member is formed of Nitinol and the second member is formed of stainless steel.

In an embodiment, the first member comprises a distal member of the medical component and the second member comprises a proximal member of the medical component, the first portion of the first member comprising a proximal end of the first member, the first portion of the second member comprising a distal end of the second member.

In an embodiment, the first and second members each comprise tubes so that the medical component comprises a unitary tube.

In an embodiment, the first and second members each comprise wires so that the medical component comprises a unitary wire.

In an embodiment, a distal end of the first member comprises a needle.

In an embodiment, the outer surface of the first portion of the first member and the outer surface of the first portion of the second member are chemically or mechanically treated to increase the static friction between the outer surfaces of the first and second members and the inner surface of the tube.

In an embodiment, the first portions of the first and second members each comprise a feature extending transversely therefrom to be received in an opening in the tube.

In an embodiment, the feature comprises a flange.

In an embodiment, the first portion of the first member and the first portion of the second member are in abutting contact.

In an embodiment, the first portion of the first member and the first portion of the second member are spaced by a gap and the tube covers the gap.

In addition, the present disclosure relates to a method for assembling a medical component. The method includes expanding a tube formed of a material comprising shape memory properties from a memorized shape comprising a first inner diameter to an expanded shape comprising a second inner diameter greater than the first inner diameter; passing the tube in the expanded shape over a first member and a second member, the first and second members having outer diameters less than the second inner diameter of the tube in the expanded shape and greater than the first inner diameter of the tube in the memorized shape; and bringing the tube to a temperature greater than a transition temperature of the material so that the tube attempts to contract to the memorized shape, the tube contracting so that an inner surface of the tube is in abutting contact with an outer surface of a first portion of the first member and an outer surface of a first portion of the second member, the tube providing a radially compressive force to the outer surfaces of the first and second members to maintain the first member in position relative to the second member at least by a static friction.

In an embodiment, the tube is expanded by a tapered mandrel press fixture.

In an embodiment, the tube is expanded in a cold bath maintained at a temperature below the transition temperature.

In an embodiment, the tube is formed of Nitinol.

In an embodiment, the first member is formed of Nitinol and the second member is formed of stainless steel.

In an embodiment, the first member comprises a distal member of the medical component and the second member comprises a proximal member of the medical component, the first portion of the first member comprising a proximal end of the first member, the first portion of the second member comprising a distal end of the second member.

In an embodiment, the first and second members each comprise tubes so that the medical component comprises a unitary tube.

In an embodiment, the first and second members each comprise wires so that the medical component comprises a unitary wire.

In addition, the present disclosure relates to a medical component which includes a first member; a second member; and an outer tube formed of a material having shape memory properties, the outer tube having a memorized shape comprising an inner diameter less than an outer diameter of the first and second members, the outer tube being positioned over an interface between the first and second members with an inner surface of the outer tube contacting outer surfaces of the first and second members so that, as the outer tube reverts toward the memorized shape, the outer tube provides a radially compressive force to the outer surfaces of the first and second members to maintain the first member in position relative to the second member.

In an embodiment, the outer tube is formed of Nitinol.

In an embodiment, the first and second members each comprise tubes so that the medical component comprises a tube having a lumen extending through the first and second members.

In an embodiment, the first and second members each comprise wires so that the medical component comprises a unitary wire.

In an embodiment, the first and second members abut against one another at the interface between the first and second members.

### BRIEF DESCRIPTION OF THE DRAWINGS

FIG. 1 shows an assembly of a component comprising a first member, a second member, and a shape-memory tube forming a compression joint to join the members according to various example embodiments.

FIG. 2 shows a first member and a second member comprising mating features for joining the members by a shape-memory tube according to various exemplary embodiments.

FIG. 3 shows an assembly of a component comprising the first member and the second member of FIG. 2 and further comprising a shape-memory tube including openings for coupling to flanges of the members according to various exemplary embodiments.

FIG. 4 shows a fixture for expanding a shape-memory tube from its original shape to an expanded shape having an expanded inner diameter according to various exemplary embodiments.

FIG. 5 shows a method for assembling a medical component comprising a shape-memory tube forming a compression joint according to various exemplary embodiments.

### DETAILED DESCRIPTION

The present disclosure may be further understood with reference to the following description and the appended drawings, wherein like elements are referred to with the same reference numerals. The exemplary embodiments describe systems and methods for assembling a medical component by joining two members by a compression joint comprising a shape-memory tube. In particular, the shape-memory tube is manufactured to have a memorized shape in which an inner diameter of the tube is approximately equal to, but some degree smaller than, an outer diameter of the two members to be joined. The shape-memory tube is then mechanically worked to expand its inner diameter to a sufficient degree such that the tube in the expanded state can be passed over the members to be joined (which may be held in abutting contact or a separated by a set distance from one another), The tube is then permitted to contract toward its memorized shape so that it provides a radially compressive force over the members forming the compression joint.

In some embodiments, the shape-memory tube is formed of Nitinol. Nickel-titanium alloys, commonly referred to as Nitinol, possess properties including a shape memory effect, superelasticity (or pseudoelasticity) and high flexibility. The shape memory effect refers to the ability of the alloy to undergo deformation and return to a predetermined shape (“remembered” shape or “memorized” shape). The shape memory effect of Nitinol is attributable to a reversable phase transformation between two different crystal structures, martensite, and austenite. The martensite phase is characterized by a distorted crystal lattice and occurs at lower temperatures, while the austenite phase is characterized by a more stable cubic crystal lattice and occurs at higher temperatures.

The transformation between martensite and austenite is triggered by changes in temperature or strain. When the material is in the austenite phase and the temperature decreases, the material transitions from the austenite finish temperature phase (A_{f}) to the austenite start temperature phase (Aₛ). As the temperature further decreases, the material transitions from the austenite phase to the R-phase according to the R_{f} temperature (the temperature at which the transformation from austenite to R-phase begins). As the temperature further decreases, the material transitions from the R-phase to the martensite phase according to the martensite start temperature (Mₛ) (the temperature at which the transformation from austenite to martensite begins) and the martensite finish temperature (M_{f}) (the temperature at which the transformation from austenite to martensite finishes). Accordingly, a Nitinol member generally transitions from a higher temperature phase to a lower temperature phase, such as the austenite phase to the martensite phase or R-phase. These transition temperatures of a nickel-titanium alloy can depend on factors including the alloy composition (e.g., ratio of nickel to titanium and/or introduction of small amounts of other elements) and heat treatment (e.g., aging or cold working).

The original (memorized) shape of a Nitinol member is set during manufacture (shape setting) and involves holding the member in the desired shape and heating the member to a predetermined temperature such as, e.g., 500 degrees C. After the original shape is set, the superelasticity of Nitinol permits a Nitinol member to undergo substantial deformation relative to its memorized shape without failure in both the martensitic phase and the austenitic phase. The shape memory effect permits the Nitinol member to recover its original shape when external forces are removed and the temperature of the member is above its A_{f} temperature, e.g., fully austenite. In the austenitic state, the Nitinol member behaves like a super spring wherein a deforming stress can be imposed on the member and the member will return to its memorized shape when the stress is removed. In the martensitic state, the Nitinol member will retain a deformed shape until the member is heated to above the A_{f} temperature.

Accordingly, Nitinol is a useful material for achieving a variety of engineering goals. In addition to the properties discussed above, Nitinol is biocompatible, corrosion resistant, and has a high strength-to-weight ratio, rendering it suitable for a variety of medical applications. Other materials commonly used for medical procedures include other metals (e.g., stainless steel, cobalt-chromium alloys, titanium, etc.), polymers (e.g., silicone, polycarbonate, PTFE, etc.), ceramics, glass, etc. Materials can be selected for the design of medical components based on considerations including biocompatibility, corrosion resistance, strength, and/or flexibility as well as the cost of the material.

Many invasive medical procedures employ long, thin members and/or devices that are introduced to the body through a bodily orifice or incision (or through the working channel of an endoscope previously introduced into the body) and advanced through body lumens under visualization to a target location. These types of members and/or devices include surgical tools (e.g., biopsy needles), catheters, and guidewires. Medical components formed of flexible materials are particularly suitable for these applications. In many cases, Nitinol is a preferred material due to its unique properties. For example, components of a biopsy needle device (e.g., a piercing end of a stylet and a catheter through which the stylet is advanced) can be formed of Nitinol. In another example, a guidewire may be formed of Nitinol. However, Nitinol is very expensive. In many cases, Nitinol components are the most expensive component of a surgical device, particularly when the component is in the form of a long wire or tube.

In some cases, the properties of Nitinol (flexibility, strength, etc.) may be important only for certain parts of such a long tube or wire such as, e.g., a distal portion of the member or any portion of a member in which enhanced flexibility may be desired. In other words, engineering design goals may be satisfied by using a component that has a distal portion formed of Nitinol and a proximal portion formed of a different material, e.g., stainless steel. Accordingly, it may be preferable (for some design purposes and/or for cost purposes) to combine materials into one component. However, joining dissimilar metals into a unitary component (e.g., a catheter, guidewire, etc.) can be challenging due to differences in properties such as melting point, thermal expansion, and chemical compatibility. Existing techniques for joining dissimilar metals include welding, brazing, soldering, adhesives, plastic heat shrink and mechanical fastening.

However, depending on the properties of the different types of metals and the applications for the joined component, these existing techniques may be inadequate. For example, it can be difficult to form a joint between a Nitinol member and a stainless-steel member that can withstand the deformative stresses applied during routine use in surgical procedures. This is especially difficult in joining hollow members having very thin walls such as, for example, needles and catheters. Furthermore, in medical applications it is extremely important that such joints do not fail within the body.

According to various exemplary embodiments, mechanisms are described for assembling dissimilar materials into a unitary component. In some embodiments, the dissimilar materials are dissimilar metals, e.g., Nitinol and stainless steel. However, the exemplary embodiments are not limited to these materials. Those skilled in the art will ascertain that a variety of different combinations of materials can be joined according to the present techniques. Additionally, two members having the same material can be joined according to the present techniques. The exemplary embodiments are generally directed to medical components including needles, catheters, guidewires, and other components having an optimized flexibility and cost.

According to various exemplary embodiments, methods are described for assembling two dissimilar materials by a radial compression joint formed of a shape-memory alloy, such as Nitinol. In some embodiments, a shape-memory tube (e.g., a Nitinol overtube) is compressed over the two dissimilar materials and the assembly is held together by static friction. In some embodiments, the overtube is formed of Nitinol. It should be understood that different variations of Nitinol (e.g., different alloy compositions and/or manufacturing techniques) may be suitable depending on design considerations, e.g., the type of component being assembled and its intended use. Additionally, other shape-memory alloys may be suitable for the described purposes depending on design considerations.

The superelastic and shape memory properties of Nitinol are leveraged to assemble the component. The Nitinol tube is manufactured to have an inner diameter (ID) slightly smaller than the outer diameters (OD) of the members to be joined. Prior to assembly, the Nitinol tube is expanded to a larger inner diameter (ID) so that it is able to fit over the two dissimilar members. The Nitinol tube is expanded to the larger ID while in the lower temperature state (martensite or R-phase at a temperature below M_{f} or R_{f}) so that it maintains the expanded shape. The dissimilar members are able to be connected to one another via the Nitinol tube that is in an expanded state. The two dissimilar members are fit inside the expanded Nitinol tube so that the tube is positioned around a portion of each of the members. After the members and the tube are positioned as desired, the assembly is then warmed to a higher temperature (greater than or equal to A_{f}) triggering transformation to austenite. In one example, the tube properties can be configured so that room temperature is greater than or equal to A_{f} such that the Nitinol tube tries to restore its original (memorized) shape at room temperature. This action will thus shrink the ID of the Nitinol tube and then radially compress against the two dissimilar components. This radial compression is sufficient to hold the assembly together via static friction.

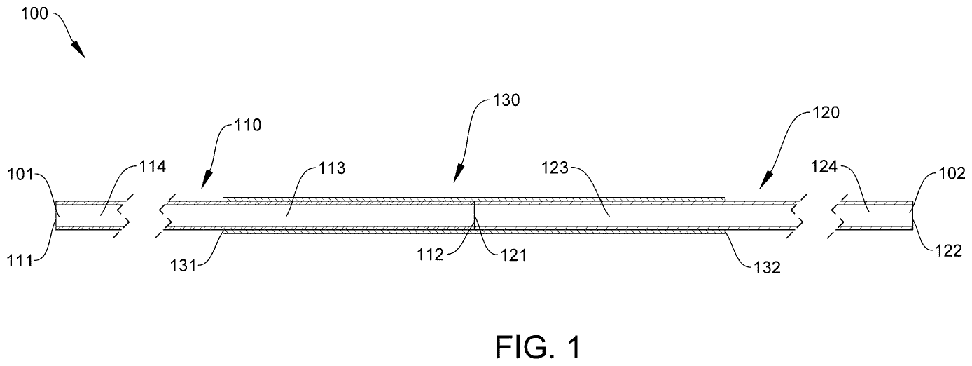

FIG. 1 shows an assembly of a component 100 comprising a first member 110, a second member 120, and a tube 130 (e.g., a shape-memory tube) forming a compression joint to join the first and second members 110, 120 according to various example embodiments. The component 100 may correspond to several different types of surgical equipment, to be described in greater detail below. In this example, the component 100 is a tube, e.g., a hypotube, a catheter, etc. Accordingly, the first member 110 comprises a first tube and the second member 120 comprises a second tube.

In other examples to be described below, the component can comprise a wire. In this example, the first member 110 and the second member 120 have similar outer diameters and similar inner diameters. In other examples to be described below, it is not required for the members to have the same OD or ID. The component 100 extends from a first end 101 to a second end 102. The first member 110 extends from a first end 111 (corresponding to the first end 101 of the component 100) to a second end 112 and the second member 120 extends from a first end 121 to a second end 122 (corresponding to the second end 102 of the component 100). In this embodiment, the second end 112 of the first member 110 is in abutting contact with the first end 121 of the second member 120. In other examples to be described below, the members can be longitudinally separated.

The tube 130 extends from a first end 131 to a second end 132. The tube 130 is positioned to overlap a first portion 113 of the first member 110 and a first portion 123 of the second member 120. In this example, the length of the overlapped first portion 113 of the first member 110 and the length of the overlapped first portion 123 of the second member 120 are equivalent, e.g., the longitudinal midpoint of the tube 130 overlaps the region where the second end 112 of the first member 110 abuts the first end 121 of the second member 120. In other examples, the overlapping regions can have different lengths. A second portion 114 of the first member 110 (including the first end 111) and a second portion 124 of the second member 120 (including the second end 122) extend out of the tube 130 and are uncovered.

The inner surface of the tube 130 is in abutting contact with the outer surface of the overlapped first portion 113 of the first member 110 and the overlapped first portion 123 of the second member 120. The tube 130 provides a radially compressive force that functions to join the first member 110 and the second member 120 into a unitary member. The first member 110 and the second member 120 are maintained in position relative to one another in this embodiment via static friction. It should be understood that the first member 110 and/or the second member 120 may deform slightly under the compressive force depending on various factors including the material, the cross section (e.g., wall thickness), and the compressive force being imposed upon the respective first and second members 110, 120. As would be understood by those skilled in the art, the compressive force, which depends on various factors including the material, the cross section, and the “memorized” shape (e.g., memorized cross-section or ID) of the tube 130, is selected so that the first and second members 110, 120, respectively, are not crushed and their internal lumens remain open.

The tube 130 is manufactured to have a memorized shape comprising an inner diameter some degree smaller than the outer diameter of the first and second members 110, 120. As described below, the tube 130 is mechanically worked so that its inner diameter is expanded to exceed the outer diameter of the first and second members 110, 120 to a degree sufficient to permit the tube 130 to be passed over the first member 110 and the second member 120. When the tube 130 is mechanically worked in a sufficiently cold setting, e.g., a cold-water bath, a liquid nitrogen bath, or cold air, the tube 130 retains this expanded shape until the tube 130 is warmed to a temperature above the A_{f} temperature.

Thus, while maintaining the temperature of the tube 130 below the A_{f} temperature, the first member 110 and the second member 120 are brought within the tube 130. The tube 130 is positioned over the first portions 113, 123 of the first and second members 110, 120 so that, as the tube 130 is warmed and compresses toward its memorized shape, the inner diameter of the tube 130 reduces toward its original ID to press against the outer diameter of the members 110, 120 applying a compressive force to the members 110, 120. The degree of compressive force is directly correlated to the memorized shape (ID) to which the tube 130 is attempting to return.

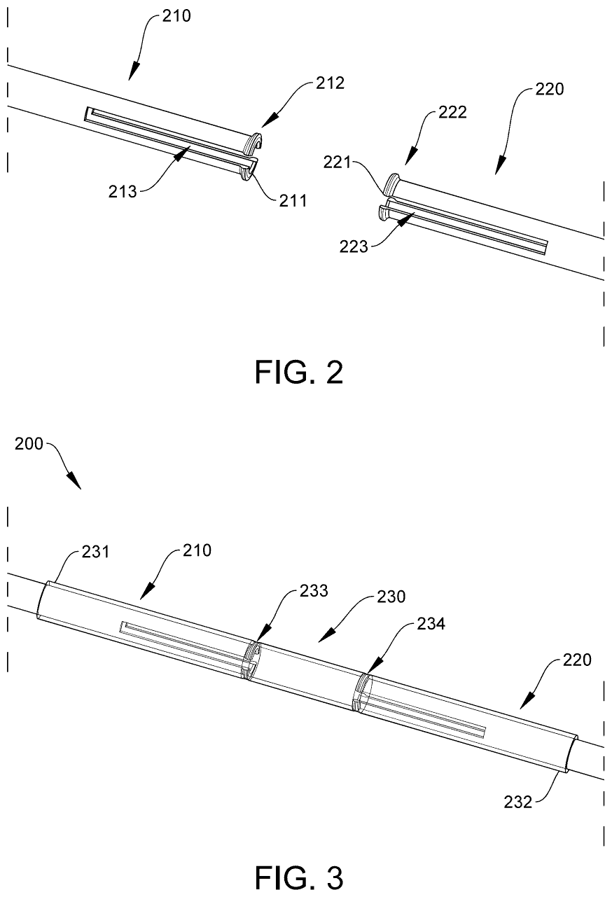

It should be understood that the component 100 described above comprises only one example. In various embodiments, the first and second members to be joined can comprise any length. Additionally, the first portions of the members (covered by the shape-memory tube) can comprise various lengths. For example, it is not required that these portions of the tube have an equal length permitting the Nitinol portions of such devices to be limited to only those portions of the device that require the particular properties of Nitinol. This reduces the overall cost of the devices while allowing them to perform in a manner similar to components made entirely of Nitinol. In some embodiments, the first and second members 110, 120 comprise wires joined into a guidewire functioning as a unitary member. In various embodiments, the members to be joined can be in abutting contact or can be spaced, e.g., as shown below in FIGS. 2-3. In various embodiments, the members to be joined can comprise various materials and are not limited to Nitinol and stainless steel.

In various embodiments, the shape-memory tube can comprise different lengths. A minimum length of the shape-memory tube can be determined, for example, based on a minimum static friction required for the application. Additionally, the shape-memory tube can comprise different wall thicknesses. In various embodiments, the memorized shape of the tube, in particular, its inner diameter, can vary with regard to the outer diameter of the members to be joined.

To increase the friction created by the radial compression of the Nitinol overtube, a variety of modifications may be made to the dissimilar members. In some embodiments, the ends of one or both of the dissimilar members (e.g., the first portions to be covered by the Nitinol tube) are chemically or mechanically treated to roughen the exterior surface to increase friction between the members. In some embodiments, mating features can be added to the components to form a tighter fit, as shown below in FIGS. 2-3.

FIGS. 2 and 3 show a first member 210 and a second member 220 comprising mating features for enhancing the joining of the first and second members 210, 220 by a tube 230 (e.g., a shape-memory tube) according to various exemplary embodiments. Similar to the component 100 of FIG. 1, the first member 210 comprises a first tube and the second member 220 comprises a second tube. The first member 210 extends from a first end (not shown) to a second end 211 and the second member 220 extends from a first end 221 to a second end (not shown). The second end 211 of the first member 210 is positioned within the tube 230 so that it is spaced a desired distance apart from the first end 221 of the second member 220. The first end of the first member 210 and the second end of the second member 220 are not shown in FIG. 2. However, as would be understood by those skilled in the art, the first and second members 210, 220 can comprise any length and the ends of the first and second members 210, 220 can comprise any shape (e.g., needle, etc.).

In this example, each of the first and second members 210, 220 comprises a flange and a slit. The first member 210 includes a flange 212 on its second end 211 and the second member 220 includes a flange 222 on its first end 221. The first member 210 includes a longitudinal slit 213 extending from the second end 211 a length toward the first end. The slit 213 extends through two sides of the first member 210 such that the first member 210 is bisected. Similarly, the second member 220 includes a longitudinal slit 223 extending a length from the first end 221 toward the second end.

FIG. 3 shows an assembly of a component 200 comprising the first member 210 and the second member 220 of FIG. 2 and further comprising the tube 230 including first and second openings 233, 234 configured for coupling to the flanges 212, 222 of the first and second members 210, 220 according to various exemplary embodiments. Similar to the component 100, the component 200 may correspond to several different types of medical equipment.

The tube 230 extends from a first end 231 to a second end 232. The tube 230 is positioned (as described above) to overlap a first portion of the first member 210 including the flange 212, the slit 213 and a length from the end of the slit 213 toward the first end of the first member 210. The tube 230 overlaps a first portion of the second member 220 including the flange 222, the slit 223 and extends a length from the end of the slit 223 toward the second end of the second member 220. The tube 230 includes the first opening 233 and the second opening 234 aligned with the flanges 212, 222. Accordingly, a gap extending longitudinally along the tube 230 is maintained between the second end 211 of the first member 210 and a first end 221 of the second member 220. It should be understood that such a gap is not required. For example, the flanges 212, 222 can be located away from the first and second ends 211, 221 of the first and second members 210, 220 so that the first and second ends 211, 221 can be brought into abutting contact with one another in a manner similar to the component 100 of FIG. 1, while the flanges 212, 222 remain spaced apart from one another.

The first member 210 and the second member 220 are positioned so that the first and second openings 233, 234 in the tube 230 are positioned over the flanges 212, 222 so that, as the tube 230 is warmed and attempts to return to the memorized shape, the flanges 212, 222 are received within the first and second openings 233, 234. The purpose of the slits 213, 223 is to enable the first and second members 210, 220 to pass into the shape memory tube 230, e.g., by pressing the opposing sides of the slits 213, 223 together to reduce the OD of the first and second members 210, 220. Without the slits 213, 223, if the OD of the first and second members 210, 220 is close to the ID of the expanded shape-memory tube 230, then it may be difficult to pass the members 210, 220 inside the tube 230 since the flanges 212, 222 may be larger in diameter than the ID of the shape memory tube 230. Accordingly, the slits 213, 223 enable the tube portions with the flange 212, 222 to pass into the shape memory tube 230 to the openings 233, 234.

It should be understood that the mating features could comprise a variety of configurations and are not limited to the arrangement shown in FIGS. 2-3. It should be understood that the principles described above can be applied in a variety of manners depending on design objectives for the medical component. Those skilled in the art will understand that in certain embodiments one of the first and second members 210, 220 may be structured to mechanically engage with a corresponding structure of the tube 230 while the other of the first and second members 210, 220 is held in position by static friction alone. Similarly, both of the first and second members 210, 220 may include structures configured to mechanically engage with a corresponding feature of the tube 230 but the structure of each of the first and second members 210, 220 (and the corresponding features of the tube 230) may differ from one another. Additionally, in embodiments where flanged members are used (such as the component 200 described above), it may not be necessary to include the slits for assembling the component as described above, e.g., if the diameter of the flanges are close to the OD of the members, or if the shape memory tube is worked such that the ID is sufficiently larger than the diameter of the flanges to permit the flanges to pass within the tube.

In one embodiment, the first member can comprise a distal member and the second member can comprise a proximal member. The first member can have a distal end comprising a needle end or any other type of distal end suitable for surgical purposes. For example, the combined component can comprise a component included in a fine needle biopsy (FNB) device that is advanced to a target anatomy under endoscopic ultrasound (EUS) or endobronchial ultrasound (EBUS)

This assembly is manufactured with fixturing that enables the Nitinol tube to be expanded in diameter. In one embodiment, a tapered mandrel press fixture forces the Nitinol tube to a larger diameter by forcing the tube over a tapered mandrel while in a cold bath (at a temperature where the lower temperature phase is thermodynamically stabile which is lower than M_{f} or R_{f}). The cold bath can be ice water, liquid nitrogen, or any other suitable setting that enables the Nitinol tube to be transformed to its lower temperature phase (Martensite or R-phase). Because the Nitinol tube will be in a cold bath, it will maintain its larger diameter shape when pulled off the tapered mandrel. This larger diameter Nitinol overtube will then be able to be fit over the two dissimilar components. After the Nitinol overtube is fit over the dissimilar components, the tube can then be removed from the cold bath and warmed up to constrict over the dissimilar components at a warmer temperature.

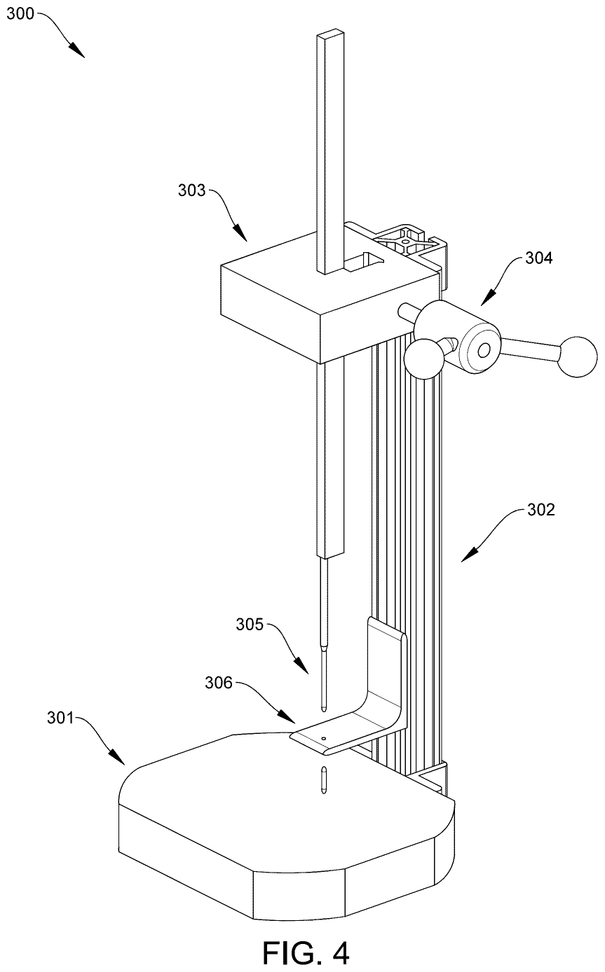

FIG. 4 shows a fixture 300 for expanding a shape-memory tube from its original shape to an expanded shape having an expanded inner diameter according to various exemplary embodiments. The fixture 300 includes a base 301, a backplate 302 extending transverse to the base 301, and a press 303 mounted to the backplate 302 above the base 301. The press 303 can be actuated by a handle 304. A mandrel 305 is fixed to the press 303. The mandrel 305 is tapered such that a smaller diameter end of the mandrel 305 is pointed toward the base 301. The mandrel 305 has at least a portion that is cone-shaped (e.g., a frustum of a cone), e.g., the diameter of the mandrel 305 increases from a first diameter to a second diameter. The mandrel 305 is coupled to the press 303 which is coupled to the backplate 302 so that the press 303 and the mandrel 305 can be translated in a first direction (e.g., down) and a second direction (e.g., up) by operating the handle 304 of the press 303.

A bracket 306 is fixed to the backplate 302. The bracket 306 is sized and shaped to hold the shape memory tube. Accordingly, the shape-memory tube can be mounted to the bracket 306 such that mandrel 305 is aligned with the ID of the tube. The press 303 can be translated toward the tube so that the smaller diameter end of the mandrel 305 accesses the ID of the shape-memory tube. The mandrel 305 can be advanced into the shape-memory tube so that the shape-memory tube is expanded to the second diameter of the mandrel 305. It should be understood that the mandrel 305 is sized to expand the shape-memory tube to an ID greater than the OD of the members to be joined as described above. After expansion, the press 303 is translated upward so that the tube can be removed from the mandrel 305 through the interference of the bracket 306.

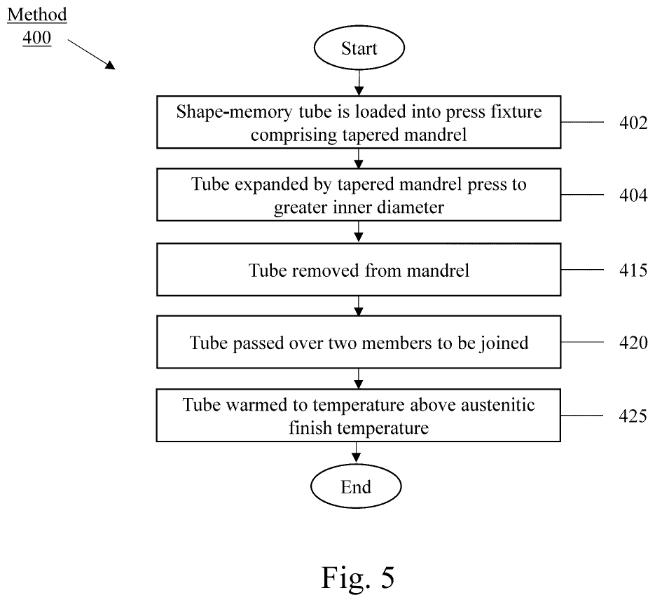

FIG. 5 shows a method 400 for assembling a medical component comprising a shape-memory tube forming a compression joint according to various exemplary embodiments. In 402, the shape-memory tube is loaded into a press fixture comprising a tapered mandrel as described above. The press fixture is at least partially submerged in a cold bath or is otherwise maintained in a cold environment where the lower temperature phase is thermodynamically stabile which is lower than M_{f} or R_{f}. In 404, the tube is expanded to a greater ID by the tapered mandrel press in the manner described above. In 406, the tube is removed from the tapered mandrel. In 408, while the temperature of the shape-memory tube remains in the lower temperature phase, the tube is passed over two members to be joined.

In 410, the shape-memory tube is then warmed to a temperature at or above its Aₛtemperature so that it transitions to its austenite phase and contracts radially into contact with the outer surfaces of the members to be joined providing a radially compressive force to these members and joining them members via static friction as described above. As would be understood by those skilled in the art, where one or more of the members to be joined and the shape-memory tube includes components configured to mechanically engage with one another, these components need only be aligned as desired in step 408.

The above embodiments are described with regard to cylindrical members being joined by a cylindrical tube. However, the exemplary embodiments are not limited to cylindrical components. For example, members having cross sections shaped differently than a circle, e.g., oval, or a non-rounded shape, can be joined according to appropriate modifications to the preceding embodiments.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims. Specifically, although this application describes various embodiments each having different features in various combinations, those skilled in the art will understand that any of the features of one embodiment may be combined with the features of the other embodiments in any manner not specifically disclaimed or which is not functionally or logically inconsistent with the operation of the device or the stated functions of the disclosed embodiments.

1-15. (canceled)

16. A medical component, comprising
a first member;
a second member; and
a tube formed of a material comprising shape memory properties, the tube having a memorized shape comprising an inner diameter less than an outer diameter of the first and second members, the tube positioned so that an inner surface of the tube is in abutting contact with an outer surface of a first portion of the first member and an outer surface of a first portion of the second member, the tube providing a radially compressive force to the outer surfaces of the first and second members to maintain the first member in position relative to the second member at least by a static friction.

17. The medical component of claim 16, wherein the tube is formed of Nitinol.

18. The medical component of claim 16, wherein the first member is formed of Nitinol and the second member is formed of stainless steel.

19. The medical component of claim 16, wherein the first member comprises a distal member of the medical component and the second member comprises a proximal member of the medical component, the first portion of the first member comprising a proximal end of the first member, the first portion of the second member comprising a distal end of the second member.

20. The medical component of claim 19, wherein the first and second members each comprise tubes so that the medical component comprises a unitary tube.

21. The medical component of claim 19, wherein the first and second members each comprise wires so that the medical component comprises a unitary wire.

22. The medical component of claim 19, wherein a distal end of the first member comprises a needle.

23. The medical component of claim 16, wherein the outer surface of the first portion of the first member and the outer surface of the first portion of the second member are chemically or mechanically treated to increase the static friction between the outer surfaces of the first and second members and the inner surface of the tube.

24. The medical component of claim 16, wherein the first portions of the first and second members each comprise a feature extending transversely therefrom to be received in an opening in the tube.

25. The medical component of claim 24, wherein the feature comprises a flange.

26. The medical component of claim 16, wherein the first portion of the first member and the first portion of the second member are in abutting contact.

27. The medical component of claim 16, wherein the first portion of the first member and the first portion of the second member are spaced by a gap and the tube covers the gap.

28. A medical component, comprising
a first member;
a second member; and
an outer tube formed of a material having shape memory properties, the outer tube having a memorized shape comprising an inner diameter less than an outer diameter of the first and second members, the outer tube being positioned over an interface between the first and second members with an inner surface of the outer tube contacting outer surfaces of the first and second members so that, as the outer tube reverts toward the memorized shape, the outer tube provides a radially compressive force to the outer surfaces of the first and second members to maintain the first member in position relative to the second member.

29. The medical component of claim 28, wherein the first and second members each comprise tubes so that the medical component comprises a tube having a lumen extending through the first and second members.

30. The medical component of claim 28, wherein the first and second members each comprise wires so that the medical component comprises a unitary wire and wherein the first and second members abut against one another at the interface between the first and second members.

31. A method for assembling a medical component, comprising:
expanding a tube formed of a material comprising shape memory properties from a memorized shape comprising a first inner diameter to an expanded shape comprising a second inner diameter greater than the first inner diameter;
passing the tube in the expanded shape over a first member and a second member, the first and second members having outer diameters less than the second inner diameter of the tube in the expanded shape and greater than the first inner diameter of the tube in the memorized shape; and
bringing the tube to a temperature greater than a transition temperature of the material so that the tube attempts to contract to the memorized shape, the tube contracting so that an inner surface of the tube is in abutting contact with an outer surface of a first portion of the first member and an outer surface of a first portion of the second member, the tube providing a radially compressive force to the outer surfaces of the first and second members to maintain the first member in position relative to the second member at least by a static friction.

32. The method of claim 31, wherein the tube is expanded by a tapered mandrel press fixture.

33. The method of claim 31, wherein the tube is expanded in a cold bath maintained at a temperature below the transition temperature.

34. The method of claim 31, wherein the tube is formed of Nitinol.

35. The method of claim 31, wherein the first member comprises a distal member of the medical component and the second member comprises a proximal member of the medical component, the first portion of the first member comprising a proximal end of the first member, the first portion of the second member comprising a distal end of the second member.
